(19) United States
(12) Patent Application Publication
(10) Pub. No.: US 2009/0018505 A1
(43) Pub. Date: Jan. 15, 2009

(54) Powered automatic injection device

(76) Inventors: Andrew Arguedas, Minneapolis, MN (US); Wilber Eidson, Minneapolis, MN (US)

Correspondence Address: Janet P. Schafer;SCHAFER LAW OFFICE, P.A., 727 Torchwood Circle, New Brighton, MN, 55112-2538, US

(21) Appl. No.: 12/215,784

(22) Filed: Jun. 30, 2008

Related U.S. Application Data
(63) Continuation-in-part of application No. 10/916,069, filed on Aug. 11, 2004, now abandoned.
(60) Provisional application No. 60/494,213, filed on Aug. 11, 2003.

(51) Int. Cl. A61M 5/20

(52) U.S. Cl. 604/131

(57) ABSTRACT

An automatically controlled medicament delivery system having a hand-held gun-shaped device with a handgrip. The device has an internal motor that is powered by connection to a power source, such as AC current or DC battery, a pair of limit switches that control the delivery of the medicament by limiting movement of a helical gear that moves a fitting in response to being powered by the internal motor, the helical gear transferring rotational motion from the electric motor into linear motion through a coupling attached to the helical gear.

This is a continuation-in-part application of Ser. No. 10/9016,069, filed Aug. 11, 2004 and is hereby incorporated by reference.

### BACKGROUND

This invention relates to veterinary delivery devices for delivering medicaments, including pharmaceuticals or vaccines, to a plurality of poultry or other animals. In particular, this invention relates to a portable electrically powered veterinary delivery system for reliably providing a precise amount of pharmaceuticals, vaccines, or an implantable device, rapidly to a plurality of animals, including fowl, porcine, ovine, bovine, piscine, or other animals.

Pox experienced by chickens, and other fowl, is a painful, fatal disease which results in damaging the poultry crop for the farmer. Numerous injection devices have been provided in prior art that are adapted to include a manually operated plunger needle or push rod. A hand-held syringe, having a barrel and a manually operated plunger, has been used to administer vaccines, antibiotics and other biological products. A problem with this method is that the accuracy of the dose is dependent on the manual strength and attention of the operator. For example, when injecting large numbers of birds or other animals, the operator's hand becomes fatigued. This results in inaccurate doses being delivered to the bird, or other animal, being injected. Additionally, this manual method of delivery of medicament is time consuming.

An additional prior method of vaccinating birds included holding a bottle of vaccine in one hand and a “pox needle”, a solid needle with an orifice formed therein, in the other hand. The operator must spread the wing of the bird, dip the needle in the bottle, stab the bird all while trying to avoid self-injection.

An alternative prior method of vaccination birds for pox is a device that requires an operator to pour the vaccine into a bottle with a “pox needle” inside. The device was similar to a syringe but without a plunger. This device has a spring-loaded handle that is connected to the “pox needle”. When the handle is compressed, the needle is pushed out through a gasket in the tip of the device. For an operator to deliver a dose to a bird, the operator must compress the spring-loaded handle until the orifice on the needle passes through the bird's wing web. This method causes fatigue to the operator's hands when vaccinating large numbers of birds and is also time consuming.

An additional problem with mass vaccination of animals has been accidental injection injury to the operator as a result of either movement of the bird, or other animal, during the injection process. Injection into a human of certain veterinary products can cause permanent injury that may result in amputation. Repetitive stress injury is also a common injury with this method of mass vaccination.

An additional problem with the prior art of mass vaccination has been keeping track of the inoculated animals so as to not administer a double dose, or inadvertently skip inoculating an animal among so many. Because of the short shelf-life of the vaccine, being able to quickly administer vaccine to a large number of birds, or other animals, is important. Some vaccines have such a short shelf-life that, once opened, the vaccine needs to be used within one hour.

While these units may be suitable for the particular purpose which they address, they would not be as suitable for the purpose of the present invention as hereinafter described.

### SUMMARY

The present invention is directed to an economic, automatically controlled medicament delivery system having a hand-held gun-shaped device that provides a rapid and consistent one-handed administration of the medicament or implantable device, without fatigue-affected changes in the amount of medicament delivered, the device rapidly delivers a precise amount of medicament to an individual animal within a multiplicity of similar animals. The device of this invention has a handgrip, with an internal motor that is powered by connection to a power source, such as AC current or DC battery. A pair of electric limit switches are provided to control the administration of the medicament by limiting movement of a helical gear that moves a fitting in response to being powered by an internal motor. The limit switches control the fitting at effectuates the administration of the medicament by means of switching polarities of the motor. Depressing the trigger on the gun-shaped device initiates the internal motor that in turn powers a helical gear transferring rotational motion from the electric motor into linear motion through a coupling attached to the helical gear. The coupling in turn drives the fitting and delivers the medicament to the individual animal. Needle safety switch, where provided, requires contact of the pressure-sensitive safety switch on the device with an animal in addition to depressing the trigger to close the electric circuit and activate the helical gear, delivering the medicament. Other safety switches, where provided, are controlled by the user to stop delivery of the medicament when the medicament is being delivered in error.

The device has four LED indicator lights that signal the operator the mode the device is in. E.g. an amber LED indicates mode one with the power on and the device is ready to inject. A green LED indicates mode two, the power is on, internal gear motion is commenced forward , injection in progress. A blue LED indicates mode three, the power is on, motion is topped and injection has been completed. A red LED indicates mode four, the power is on, motion is reversed and the injection device is automatically being primed for the next injection.

The device has optional agencies of delivering the medicament, these agencies being a solid needle, a hollow needle for an injectable, a hollow needle for subcutaneous insertion of an implantable object, and a spray nozzle for spraying medicament in nasal passages or administering a fluid externally to the animal. This device provides a rapid and consistent one-handed administration of the medicament or implantable device, without fatigue-affected changes in the amount of medicament deliver.

An automatic veterinary medicament delivery system is provided that satisfies these needs for delivery, in quick succession, precise amounts of medicament, or an implantable device, to a multiplicity of individual animals without injury to the worker, either by injection in error or by repetitive stress injury. A pressure-sensitive needle safety switch is provided adjacent to the needle, pressure on this safety switch initiates forward motion of the motor. Injection cannot take place until the needle is fully inserted, thus enabling the operator to withdraw an accidental stab prior to injection taking place. The safety switch and the trigger must be activated to complete the circuit so injection can take place. Additionally, by constantly pulling the trigger, the user can rapidly inject, e.g. poultry, by pressing safety switch against the body of the bird which closes the circuit and injection takes place. By pulling the trigger once and touching the device against the body of the animal to be injected, multiple animals (birds) are being injected rapidly with the precise dosage. This feature relieves repetitive stress injuries to the worker because they are not repeatedly depressing the trigger. A goal of the particular system is operator safety. Self-injection is a very serious accident among vaccinating crews as is repetitive stress injury.

### BRIEF DESCRIPTION OF THE DRAWINGS

Understanding the invention will be enhanced by referring to the accompanying drawings, in which:

FIG. 1A is a side perspective view with certain portions shown in cross-section of the hand-held powered automatic injection device of this invention , with the needle extended;

FIG. 1B is a side perspective view, with certain portions shown in cross-section, of the device of FIG. 1A with the needle retracted;

FIG. 2 is a side perspective view of the hand-held unit with certain portions shown in cross-section;

FIG. 3 is a side perspective view of the hand-held unit with certain portions shown in cross-section; and

FIG. 4 is a side perspective view of the hand-held unit with certain portions shown in cross-section.

### DETAILED DESCRIPTION OF CURRENTLY PREFERRED EMBODIMENTS

The understanding of the invention will be further enhanced by referring to the following illustrative but non-limiting examples.

The term “medicaments” is intended to include serum, vaccine, antibiotic and other fluid products and pelletized products, such as hormones or identification information, that may be used for immunizing, medically treating, identifying, animals, such as poultry, bovine, porcine, piscine or other animals. The term “poxer needle” is intended to include a solid needle having an orifice formed in the side thereof.

Turning now to the drawings, in which like numbers refer to corresponding elements throughout several views, shown is an automatic electrically powered hand-held gun-shaped device with a handgrip, the device of unitary construction. The injection device has an internal motor that is powered by connection to a power source, such as AC current or DC battery. The injection device is activated when the trigger on the injection device handle is manually depressed which closes an electrical circuit and initiates the internal motor to drive a helical gear that moves a fitting that results in the delivery of the medicament, or the like, to the individual animal. The fitting is limited in movement by a pair of limit switches which automatically reverses the motion of the fitting, preparing the device for the next administration of a medicament or the like. The injection device has indicator lights that signal the operator when the injection device is ready to deliver the medicament, when the medicament is being delivered, when the medicament has been delivered, and when the helical gear and associated fitting is being returned to the original position readying the injection device for the next delivery of medicament, or the like. Additionally, a needle safety switch is provided to add a safety feature in that the needle safety switch must be in contact with an animal when the trigger is depressed to close the electrical circuit to activate the helical gear.

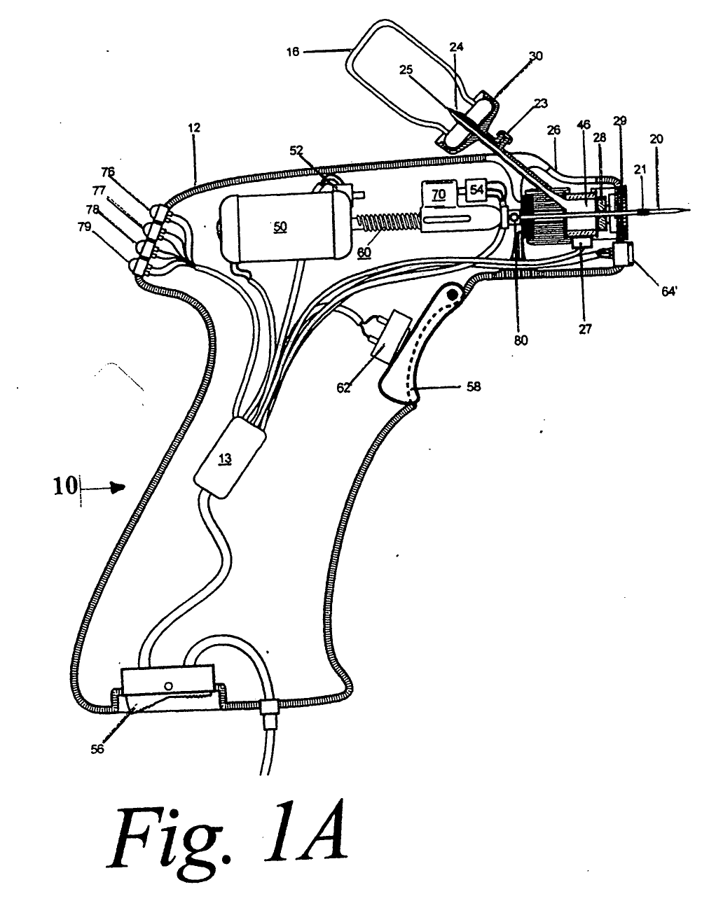

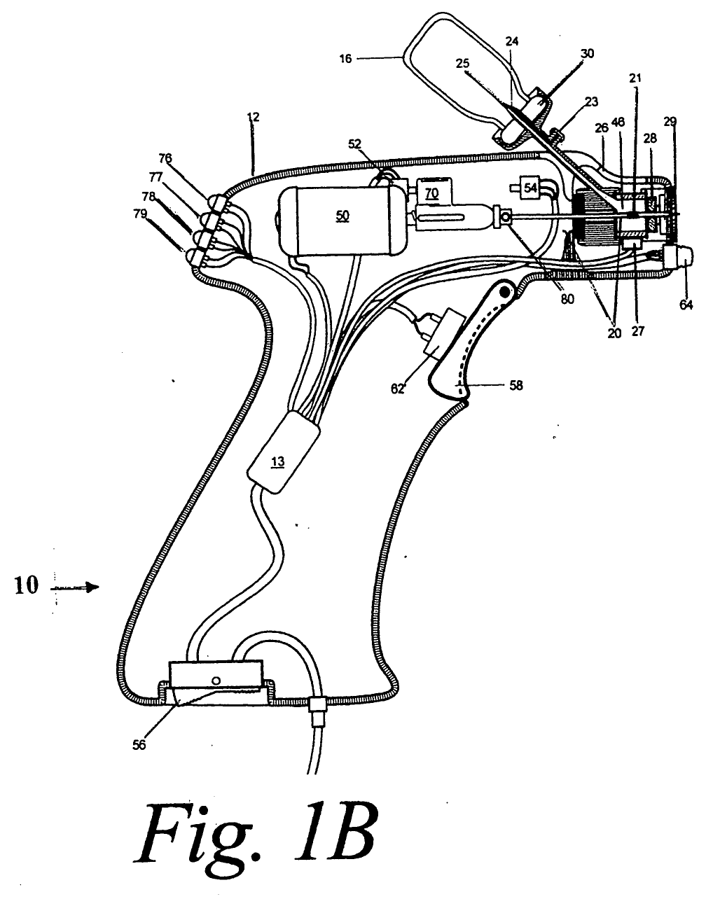

FIGS. 1A & 1B illustrate an automatic electrically powered hand-held medicament delivery device 10 having a gun-shaped body 12 with a single solid needle 20. Motor 50 is an electric motor held internally to the hand-held device that is powered by connection to a power source, such as AC current or DC battery. Because a simple motor is used, the cost of manufacturing is less than that with a computerized system. Medicament is in fluid communicate between a reservoir 16 and the medicament delivery system, here a poxer needle. The medicament flows into holding cylinder 46 and the poxer needle passes therethrough. An orifice 21, formed in the side of the poxer needle 20, receive the medicament and is pushed by the solid poxer needle 20 through the animal body part, e.g. the wing of a fowl. The poxer needle 20 is mounted on a fitting 70 and moves horizontally backwards and forwards in response to the pulling of the trigger and the depressing of the needle safety switch 64. It is important to note that both the trigger and the needle safety switch 64 must be depressed before the electrical circuit is closed and the needle carrying fitting 70 moves. Dosage is controlled by the size of the orifice 21. The poxer needle 20 s thrust so that it pierces the skin f the subject animal. When used with fowl, the poxer needle 20 would continue through the wing of the fowl leaving the medicament at the wound site. The needle 20 thrust is controlled by the electric motor 50 which turns a helical gear 60 angularly which threads into a fitting 70. The fitting 70 is attached to the solid needle 20 by means of a setscrew 80. Movement of the fitting 70 limits the trust of the needle 20. The movement of the fitting 70 is in turn limited by a pair of limit switches 52, 54 that are pre-set to limit the distance the fitting 70 travels. The pressure-sensitive safety switch 64 adjacent the needle 20 , when depressed, initiates the forward motion of the motor 50 when the trigger 58 also is depressed. This needle safety switch 64 feature also deters accidental self-injection because the medicament is not delivered until the needle is fully inserted with the needle safety switch coming into contact with the animal body to close the electric circuit, thus enabling the operator to withdraw an accidental stab prior to delivery of the medicament taking place. Repetitive stress injury is limited because the operator can constantly pull the trigger, rather than repeatedly pulling the trigger, and press the safety switch against the body of a series of birds, closing the electric circuit and delivering medicament.

Indicating lights, 76, 77, 78, 79, mounted on the handgrip 14, display to the operator the real time status of the operation mode. While the main power switch 56 is on, a holding cylinder indicating light 27 illuminates the holding cylinder 46 displaying the content of the holding cylinder 46 to the operator by the viewing port 26.

Precision dosing with the solid needle unit is controlled by the size of the orifice 21 formed in the poxer needle 20. Thrust of the poxer needle 20 is controlled by the forward limit switch 54, and the reverse limit switch 52. The distance between the two limit switches 52,54 accurately controls the poxer needle 20 thrust because they control the distance the poxer needle 20 moves. During normal operation, the needle thrust is displayed when the trigger safety switch 62 is in the depressed position, as shown in FIGS. 1A & 1B. One of the four indicating lights 76,77,78,79 illuminates displaying one of four modes to the operator noting delivery of the doses. Mode one is indicated by here an amber light 76, although other colors are able to be used provided there are discernable differences between them, representing the injection device is ready to deliver the medicament. A green light 77 indicates mode two representing the forward thrust of the delivery device; mode three is indicated by a blue indicating light 78 representing that the forward thrust is complete and ready to reverse; mode four is indicated by a red light 79 representing the reverse thrust of the poxer needle 20.

Mode one is commenced when the power switch 56 is on, the trigger safety switch 62 is depressed, the needle safety switch 64 is not depressed, and reverse limit switch 52 is depressed, illuminating only the amber indicating light 76.

Mode two is commenced when the power switch 56 is on, the trigger safety switch 62 is depressed and the needle safety switch 64′ is depressed, illuminating the green indicating light 77, and turning off the amber indicating light 76.

Mode three is commenced when the power switch 56 is on, the trigger safety switch 62′ is depressed, the needle safety switch 64′ is depressed, and the forward limit switch 54 is depressed, illuminating the blue indicating light 78 and turning off the green indicating light 77.

Mode four is commenced when the power switch 56 is on, the trigger safety switch is depressed 62″, needle safety switch 64 is not depressed, and reverse stop switch 52 is not depressed, illuminating the red indicating light 79, and turning off the blue indicating light 78. The indicator lights work similarly in all units of the device.

Medicament is held inside holding cylinder 46, where the orifice 21 is submerged in the medicament and thus fills the orifice 21. During normal operation, medicament is supply to the holding cylinder 46 by means of a drop off fitting 30, which delivers the medicament down the fluid intake port 25. During normal operation, as the level in medicament reservoir 16 is depleted, it is replaced by air by means of an intake air port 24, which only flows inward due to an intake air valve 23.

Holding cylinder 46 is sealed by an o-ring 28, which makes a seal around the poxer needle 20. During operation, when the poxer needle 20 moves outward from the holding cylinder 46, the o-ring 28 is flexible enough not to let excess medicament out by expanding with the contours to the curves of the needle orifice while the poxer needle 20 passes by the o-ring 28. Holding cylinder 46 and the o-ring 28 are held in place by a threaded cap 29.

All wire terminations and unions are made inside a sealed junction box 13. The distance between the two limit switches 52, 54 accurately controls needle thrust to render precise doses. Precision dosing and needle travel are important and is controlled during normal operation by the forward limit switch 54, and the reverse limit switch 52. These limit switches 52, 54 work by reversing polarity that reverses the direction of movement of the fitting 70. Ultimately, the thrust of the poxer needle 20 is determined by the position of the limit switches, 52, 54.

Built in safety features include: main power switch 56, which terminates any contact between an electromotive force and trigger safety switch 62; trigger safety switch 62, acts as an emergency stop button that stops all thrust of the motor 50 instantly when released; needle safety switch 64 is a switch that reverses the thrust of the poxer needle 20, when released or depressed, by changing the direction of current to the motor 50, this can occur if the device is withdrawn from the injection site at any point during injection. FIG. 1B illustrates the injection device of FIG. 1A with the needle withdrawn into the injection device 10 and the orifice is withdrawn into the holding cylinder 46 re-filling the orifice with medicament.

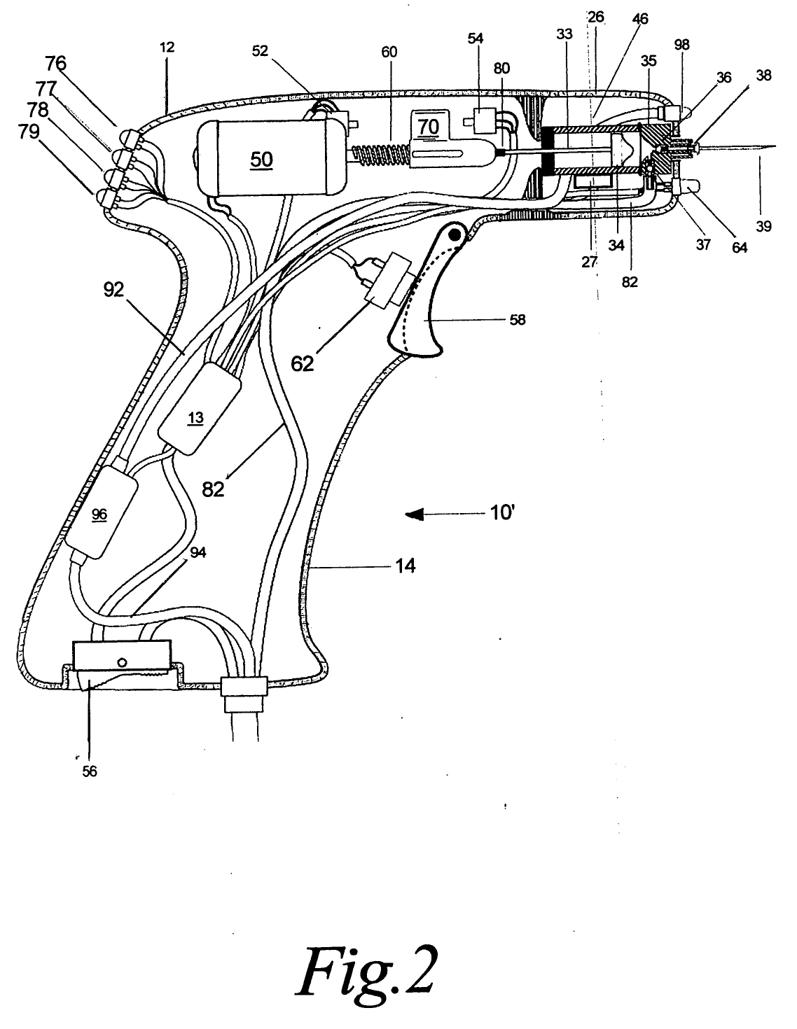

The hand-held unit 10′ is illustrated at FIG. 2 which shows an automatic electrically powered hand held medicament delivery system with a fixed single hollow needle 39. Medicament is delivered through the hollow needle 39, by a piston 34 moving in response to the depressing of the trigger 58, causing movement of the helical gear 60 to turn angularly into the fitting 70. The piston 34 is housed inside a holding cylinder 46, and is connected to fitting 70, by means of a push rod 33, and secured by a set screw 80. The piston's 34 thrust is controlled by an electric motor 50. Here the hollow needle 39 doesn't move; rather the piston held within the injection device 10 moves pushing the precisely dosed medicament held in the holding chamber 46 through the hollow needle 39 and is injected into the subject animal.

The hand-held unit 10′ again has four illuminating indicating lights to display to the operator the real time status of this operation mode. While the main power switch 56 is on, a cylinder indicating light 27, illuminates the holding cylinder 46 displaying the content of the cylinder to the operator by the viewing port 26.

Precision dosing and piston thrust is important and is stopped during normal operation by the forward limit switch 54, and the reverse limit switch 52. The distance between the two limit switches 52,54 accurately controls piston 34 thrust. During normal operation, piston 34 thrust is displayed when the trigger safety switch 62 is in the depressed position, one of the four illuminating lights illuminates displaying one of four modes to the operator assuring accurate doses. Mode one is indicated by a amber light 76 representing the injection device is ready to inject; a green light 77 indicates mode two representing the forward thrust of the piston; mode three is indicated by a blue indicating light 78 representing forward thrust is complete and ready to reverse; mode four is indicated by a red light 79 representing the reverse thrust of the piston 34. Mode one is commenced when the power switch 56 is on, the trigger safety switch 62 is depressed, the needle safety switch 64 is not depressed, and reverse stop switch 52 is depressed illuminating only the amber indicating light 76. Mode two is commenced when the power switch 56 is on, the trigger safety switch 62 is down, and the needle safety switch 64 is depressed, illuminating the green indicating light 77, and turning off the amber indicating light 76. Mode three is commenced when the power switch 56 is on, the trigger safety switch 62 is depressed, the needle safety switch 64 is depressed, and the forward limit switch 54 is depressed, illuminating the blue indicating light 78 and turning off the green indicating light 77. Mode four is commenced when the power switch 56 is on, the trigger safety switch 62′ is depressed, needle safety switch 64 is not depressed, and reverse stop switch 52 is not depressed, illuminating the red indicating light 79, and turning off the blue indicating light 78.

In FIG. 2, medicament is again held inside holding cylinder 46, where the flow of liquid medicament of controlled by two check valves housed in valve body 35. When the piston 34 moves in forward direction, exit valve 36 opens, and entrance valve 37 closes due to the positive pressure inside the holding cylinder 46. When the piston 34 is in the reverse direction, a negative pressure is caused inside the holding cylinder 46. Negative pressure inside the holding cylinder 46 causes entrance valve 37 to open, and exit valve 36 to close.

During normal operation, medicament is supplied to the holding cylinder 46 by means of flexible medicament supply tubing 82, which delivers the medicament from the reservoir external to the injection device to the intake valve 37 on the valve 35.

The hollow needle 39 is easily replaceable by the means of a luer lock 38 release system. The luer lock 38 secures the hollow needle 39 with the ¼ turn of the needle. The luer lock 38 functions as part of the valve 35.

All wire terminations and unions are made inside a sealed junction box 13. The distance between the two limit switches 52, 54 accurately controls piston 33 thrust to render precise doses. Precision dosing and piston travel are important and is controlled during normal operation by the forward limit switch 54, and the reverse limit switch 52. Ultimately the thrust of the piston is determined by the position of the limit switches 52, 54. Built in safety features include: main power switch 56, which terminates any contact between an electromotive force and device; trigger safety switch 62, acts as an emergency stop button that stops all thrust of the motor 50 instantly when released; and needle safety switch 64 is a switch that reverses the thrust of the piston 34, when released or depressed, by changing the direction of current to the motor 50, this can occur if the device is withdrawn from the injection site at any point during injection.

The hand-held unit 10′ is equipped with an optional timed dye marking system, pump 96, by way of dye tubing 92 to dye spray port 98 to identify injections sites when the injection is completed. This is an important tool for management, for monitoring the work of injection crews.

The dye marking system is run by a pump that is turned on only while in the mode three. The dye marking system is comprised of a dye reservoir external to the injection device, entrance port on the bottom of the handle; an electric motor; a pump 96 driven by the electric motor; two tubing connections on the pump; a dye spray port 98 on the front side of the of the device 10; tubing 94 connecting exit port to pump 96 intake port; tubing 92 connecting pump exit port to dye spray port 98; a spray fitting to thread into the spray port yielding a prominent spray.

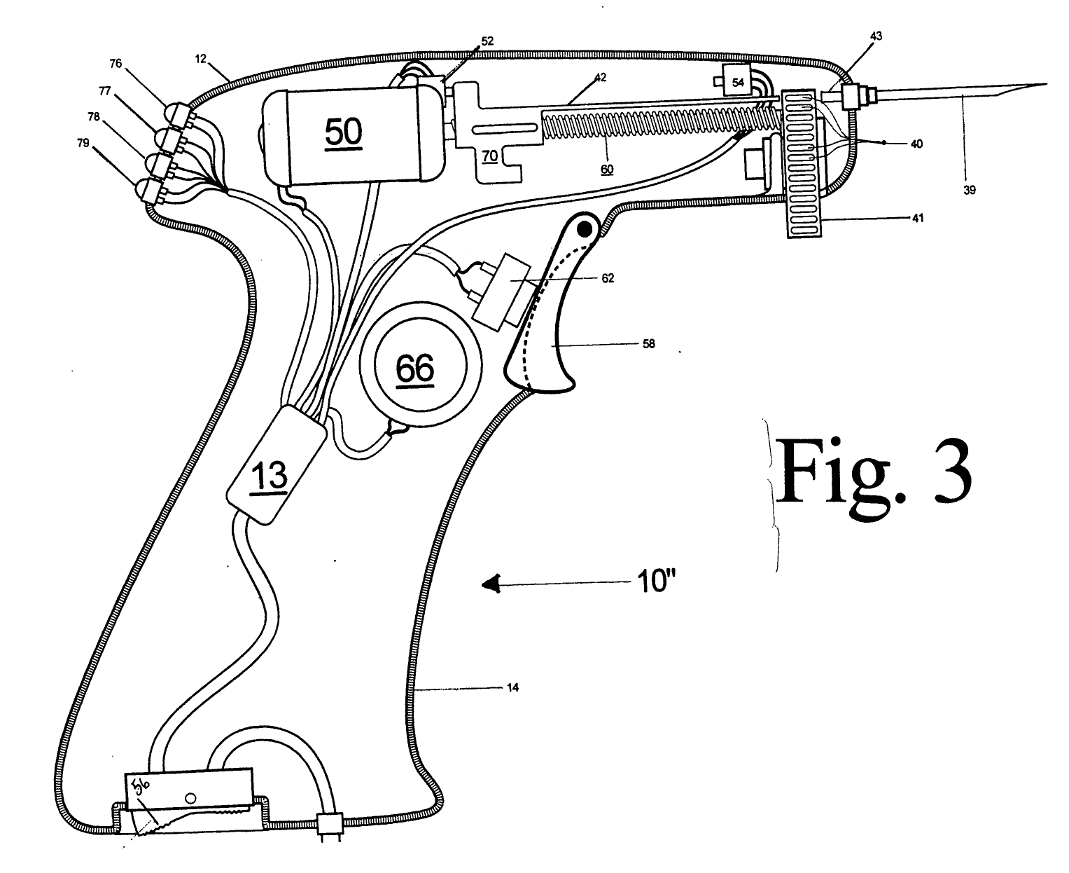

A injection device 10″ is illustrated at FIG. 3, which again is an injection device 10″ with a single hollow needle 39. Instead of delivering medicament by injection, this embodiment delivers a medicament by means of an implant 40. The implant 40 is delivered through the hollow needle 39, by a push rod 42 in response to the depressing of trigger 58. Trigger 58 is the polarity switch for motor. The push rod 42 is housed inside a guide 43, and is connected to fitting 70. The push rod 42 thrust is controlled by an electric motor 50 which turns a helical gear 60 angularly until it threads into the fitting 70. This embodiment 10″ has four indicating lights, 76,77,78,79 to display to the operator the real time status of his operation mode.

Precision dosing and push rod 42 thrust is important and is stopped during normal operation by the forward limit switch 54, and the reverse limit switch 52. The distance between the two limit switches 52, 54 accurately controls push rod 42 thrust. During normal operation, push rod 42 thrust is displayed when the trigger safety switch 62 is in the depressed position, one of the four illuminating lights illuminates displaying one of four modes to the operator assuring accurate doses. Mode one is indicated by a amber light 76 representing the injection device is ready to inject; a green light 77 indicates mode two representing the forward thrust of the pushrod; mode three is indicated by a blue indicating light 78 representing forward thrust is complete and ready to reverse; mode four is indicated by a red light 79 representing the reverse thrust of the pushrod 42. These modes operate as previously discussed.

The implant 40 is held inside a cartridge 41, where individual implants 40 are housed in separate chambers. The implant diameter is equal to or less than the diameter of the cartridge 41. The implant diameter is less than that of the hollow needle 39. The push rod 42 pushes the implant 40 out of the cartridge 41 and through the hollow needle 39 where the implant 40 exits the hollow needle 39 at the very tip. The trust of the push rod 42 does not exceed the length of the hollow needle 39. Movement of pushrod 42 is limited by limit switches 52, 54 driven by motor 50 . The forward thrust of the push rod 42 causes the cartridge 41 to move to the next the holding cell prior to the push rod 42 entering the holding cell. The hollow needle 39 is easily replaceable by means of a nut to secure the hollow needle 39 to the body of the device 10″.

All wire terminations and unions are made inside a sealed junction box 13. The distance between the two limit switches 52, 54 accurately controls push rod 42 thrust to render precise doses. Precision dosing and needle travel are important and is controlled during normal operation by the forward limit switch 54, and the reverse limit switch 52.

Ultimately the thrust of the pushrod 42 is determined by the position of the control switches. Built in safety features include: main power switch 56, which terminates any contact between an electromotive force and the device; trigger safety switch 62, acts as an emergency stop button that stops all thrust of the motor instantly when released; needle safety switch 64 is a switch that reverses the thrust of the pushrod 42, when released or depressed, by changing the direction of current to the motor 50, this can occur if the device is withdrawn from the injection site at any point during injection.

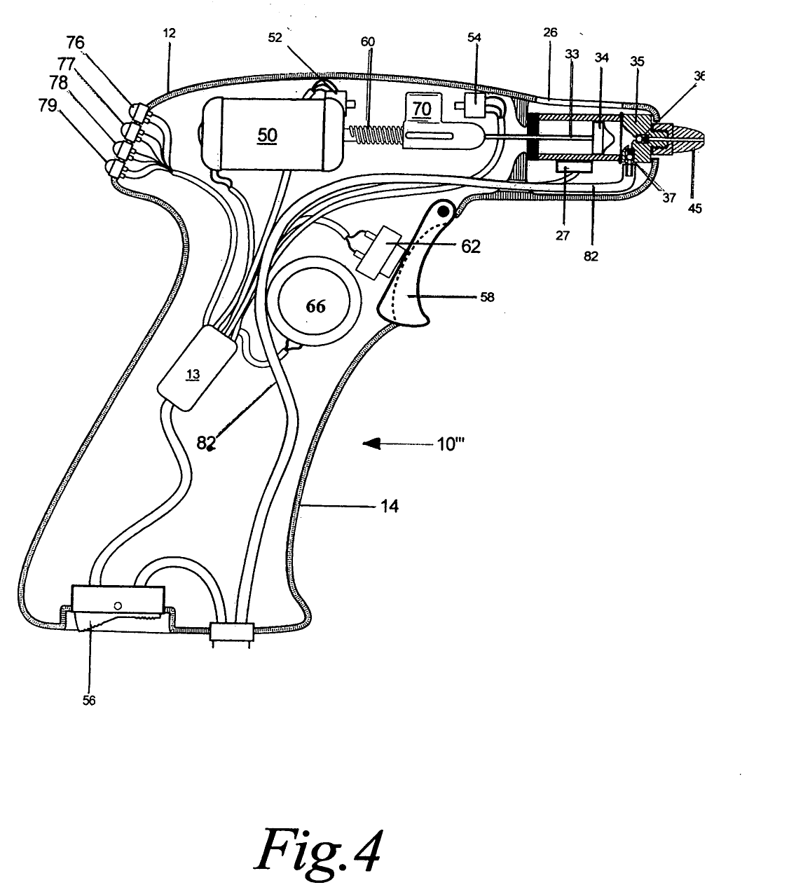

A hand-held electrically powered hand held medicament delivery system 10′″ having a spray nozzle 45 for delivery of a medicament is illustrated at FIG. 4. Here the hand-held unit 10′″ of the device again includes an internal portable power supply. Medicament is delivered through the spray nozzle 45, by force from a piston 34 in response to initiating the device by depressing the trigger 58. Trigger 58 switches the polarity of the motor again while limit switches 52, 54 limit movement of the pushrod 33. The piston 34 is housed inside a holding cylinder 46, and is connected to fitting 70, by means of a push rod 33, and secured by a set screw. The piston's thrust is controlled by an electric motor 50 which turns a helical gear 60 angularly which threads into the fitting 70.

This hand-held unit 10′″ again has four illuminating indicating lights, 76,77,78,79 to display to the operator the real time status of his operation mode. While the main power switch 56 is on, a holding cylinder 46 indicating light 27 illuminates the holding cylinder 46 displaying the content of the holding cylinder to the operator by the viewing port 26.

Precision dosing and piston thrust is important and is stopped during normal operation by the forward limit switch 54, and the reverse limit switch 52. The distance between the two limit switches 52, 54 accurately controls piston 34 thrust. During normal operation, piston 34 thrust is displayed when the trigger safety switch 62′ is in the depressed position, one of the four illuminating lights illuminates displaying one of four modes to the operator assuring accurate doses as described above. Mode one is indicated by a amber light 76 representing the injection device is ready to inject; a green light 77 indicates mode two representing the forward thrust of the piston; mode three is indicated by a blue indicating light 78 representing forward thrust is complete and ready to reverse; mode four is indicated by a red light 79 representing the reverse thrust of the piston.

Medicament is held inside cylinder 46, where the flow of liquid medicament of controlled by two check valves housed in valve body 35. When the piston thrust is in the forward direction exit valve 36 opens, and entrance valve 37 closes due to the positive pressure inside the cylinder. When the piston 34 thrust is in the reverse direction, a negative pressure is caused inside the cylinder 46. Negative pressure inside the cylinder causes entrance valve 37 to open, and exit valve 36′ to close.

During normal operation, medicament is supplied the holding cylinder 46 by means of flexible medicament supply tubing 82 which delivers the medicament to the entrance valve 37 on the valve 35.

All wire terminations and unions are made inside a sealed junction box 13.

The distance between the two limit switches 52,54 accurately controls piston 34 thrust to render precise doses. Precision dosing and piston travel are important and is controlled during normal operation by the forward limit switch 54, and the reverse limit switch 52.

Ultimately the thrust of the piston 34 is determined by the position of the control switches 52,54. Built in safety features include: main power switch 56, which terminates any contact between an electromotive force and fourth embodiment of the device. Trigger safety switch 62, acts as an emergency stop button that stops all thrust of the motor 50 instantly when released; needle safety switch 64 is a switch that reverses the thrust of the piston 34, when released or depressed, by changing the direction of current to the motor, this can occur if the device is withdrawn from the injection site at any point during injection.

We claim:

1. An economic, automatically controlled medicament delivery system for providing a rapid and consistent one-handed administration of the medicament, comprising:
a) hand-held gun-shaped housing;
b) a motor held internally by the housing, said motor is powered by connection to a power source, such as AC current or DC battery;
c) a source of medicament;
d) means for delivering a precise amount of medicament to an individual animal within a multiplicity of similar animals;
e) a trigger positioned, on said housing, whereby manually depressing said trigger on said gun-shaped device initiates said motor for powering delivery of the medicament;
f) agencies of delivering the medicament to the subject animal; and
g) one of a group of safety switches, provided externally to said housing, one for stopping the delivery of the medicament to prevent injury to the operator, and a second safety switch as a pressure-sensitive safety switch to close the electric circuit and activate said means for delivery of the medicament.

2. The device of claim 1, wherein said means delivering a precise amount of medicament to an individual animal within a multiplicity of similar animals further comprises:
a) a helical gear mounted within said housing;
b) a fitting, mounted on said helical gear, withing said housing, said fitting driven in response to said helical gear;
c) a pair of limit switches, mounted internally of said housing, that limit the movement of and automatically reverses the motion of said fitting, resulting in delivery of a pre-determined amount of the medicament.

3. The device of claim 2, wherein said agencies of delivering the medicament to the subject animal further comprise, one of a group including: a solid needle, a hollow needle for an injectable medicament, a hollow needle for subcutaneous insertion of an implantable object, and a spray nozzle for spraying medicament in nasal passages or administering a fluid externally to the animal.

4. The device of claim 3, further comprising LED Indicator lights that signal the operator the mode the device is in: the power on and the device is ready to inject; the power is on, said internal helical gear motion is commenced forward, and injection in progress; the power is on, internal helical gear motion is stopped and delivery of the medicament has been completed; and the power is on, internal helical gear motion is reversed and the injection device is automatically being primed for the next delivery.

5. An economic, automatically controlled medicament delivery system, comprising;
a) hand-held gun-shaped housing;
b) means for providing a rapid and consistent one-handed administration of the medicament;
c) means for delivering a precise amount of medicament to an individual animal within a multiplicity of similar animals;
d) a motor held internally by the housing, said motor is powered by connection to a power source, such as AC current or DC battery;
e) a source of medicament;
f) means for controlling the administration of a medicament;
g) a trigger positioned on said housing, whereby manually depressing said trigger on said gun-shaped device initiates said motor;
h) a helical gear, held internally by the housing;
i) a fitting, mounted on said helical gear, within said housing, said fitting driven in response to powered movement of said helical gear;
j) a pair of limit switches, mounted internally of said housing, that limit the movement of and automatically reverses the motion of said fitting, resulting in delivery of a pre-determined amount of the medicament
k) agencies of delivering the medicament to the subject animal; and
l) one of a group of safety switches, provided externally to said housing, one for stopping the delivery of the medicament to prevent injury to the operator, and a needle safety switch as a pressure-sensitive safety switch to close the electric circuit and activate said means for delivery of the medicament, said safety switches for preventing injury to the operator.

6. The device of claim 5, wherein said agencies of delivering the medicament to the subject animal further comprise, one of a group including: a solid needle, a hollow needle for an injectable medicament, a hollow needle for subcutaneous insertion of an implantable object, and a spray nozzle for spraying medicament in nasal passages or administering a fluid externally to the animal.

7. The device of claim 5, wherein said needle safety switch further comprises, a pressure-sensitive safety switch on the device, which when held in contact with an animal, in addition to depressing said trigger, closes the electric circuit and activates said helical gear, delivering the medicament.

8. The device of claim 5, wherein said safety switch further comprises an external safety switch and is controlled by the user to stop delivery of the medicament when the medicament is being delivered in error.

9. The device of claim 6 wherein said solid needle further comprises a poxer needle having a specially sized orifice, said orifice controlling the dosage of the medicament delivered by the device.

10. The device of claim 9, wherein said poxer needle receives the medicament that flows into a holding cylinder formed within said housing, said holding cylinder permits said poxer needle to passes therethrough, said orifice receives the medicament and delivery of the medicament occurs as said needle is pushed animal body part

11. The device of claim 10, wherein said poxer needle is mounted on said fitting and moves horizontally backwards and forwards in response to the manually depressing of said trigger and the depressing of said needle safety switch, depressed by coming into contact with the subject animal body, closing the electrical circuit such that said needle carrying fitting moves.

12. The device of claim 11, wherein movement of said fitting limits the thrust of said needle, the movement of said fitting is in turn limited by said pair of limit switches that are pre-set to limit the distance the fitting travels.

13. The device of claim 12, further comprising a pressure-sensitive safety switch mounted externally adjacent said solid needle , said a pressure-sensitive safety switch, when depressed, initiates the forward motion of said motor when said trigger also is depressed, for deterring accidental self-injection.

14. The device of claim 13, further comprising means for delivering medicament to a series of animals, said means include depressing said trigger continuously and by fully inserting said needle with said needle safety switch coming into contact with the series of animal bodies, closing the electric circuit each time said needle is fully inserted .

15. The device of claim 14, wherein continuously depressing said trigger enables the operator to withdraw an accidental stab prior to delivery of the medicament because delivery only occurs when the needle is fully inserted and by continuously depressing said trigger, the operator avoids repetitive stress injury.
